(19) United States
(12) Patent Application Publication
(10) Pub. No.: US 2013/0218178 A1
(43) Pub. Date: Aug. 22, 2013

(54) MEDICAL FABRIC WITH INTEGRATED SHAPE MEMORY POLYMER

(71) Applicants: Robin Shandas, Boulder, CO (US); Craig Joseph Lanning, Denver, CO (US); Robyn Grace Yeon Glang, Colorado Springs, CO (US); Bryan Andrew Rech, Boulder, CO (US); Naresh Mandava, Denver, CO (US); Malik Kahook, Denver, CO (US); Nageswara Rao Mandava, Jamaica, NY (US)

(72) Inventors: Robin Shandas, Boulder, CO (US); Craig Joseph Lanning, Denver, CO (US); Robyn Grace Yeon Glang, Colorado Springs, CO (US); Bryan Andrew Rech, Boulder, CO (US); Naresh Mandava, Denver, CO (US); Malik Kahook, Denver, CO (US); Nageswara Rao Mandava, Jamaica, NY (US)

(73) Assignees: The Regents of the University of Colorado, A Body Corporate, Denver, CO (US); Nageswara Rao Mandava, Jamaica, NY (US)

(21) Appl. No.: 13/821,907

(22) PCT Filed: Sep. 12, 2011
(86) PCT No.: PCT/US11/51239

§ 371 Date: Apr. 18, 2013

Related U.S. Application Data
(60) Provisional application No. 61/381,735, filed on Sep. 10, 2010.

(51) Int. Cl. A61F 2/00

(52) U.S. Cl. CPC A61F 2/0063
USPC 606/151

(57) ABSTRACT

Formulations of shape memory polymer (SMP) are integrated with several existing clinically available medical fabrics. The SMP portion of a SMP-integrated fabric can be fabricated in varying thicknesses with the minimum thickness determined by the thickness of the underlying fabric and up to almost any thickness. A large variety of patterns may be formed in SMP-integrated fabrics based upon how the shape memory polymer is integrated into the base fabric. Integration of the SMP with the base fabrics does not alter the shape memory functionality of the SMP. The design tools for controlling activation rate for traditional SMP materials thus apply to SMP-integrated fabrics. SMP-integrated fabrics may also be steam sterilized without loss of shape memory functionality. By using multiple formulations of SMP on a single piece of fabric, a large combination of material properties may be provided within a single SMP-integrated fabric device.

### CROSS REFERENCE TO RELATED APPLICATIONS

The present application claims priority under 35 USC §119(e) to U.S. Provisional Application No. 61/381,735 filed 10 Sep. 2010 and entitled “Medical fabric with integrated shape memory polymer” which is hereby incorporated herein by reference in its entirety.

The present application is related to the following applications: U.S. patent application Ser. No. 12/295,594 filed 30 Sep. 2008 entitled “Shape memory polymer medical devices”; Patent Cooperation Treaty Application No. PCT/US2006/060297 filed 27 Oct. 2006 entitled “A polymer formulation, a method of determining a polymer formulation, and a method of determining a polymer fabrication”; and U.S. patent application Ser. No. 12/988,983, filed 5 Jan. 2011 (371 date) and entitled “Thiol-vinyl and thiol-yne systems for shape memory polymers,” each of which is hereby incorporated herein by reference in its entirety.

### TECHNICAL FIELD

The technology described herein relates generally to surgical or medical repair materials and more specifically to the use of shape memory materials in surgical or medical repair materials.

### BACKGROUND

Shape memory materials are defined by their capacity to recover a predetermined shape after significant mechanical deformation. The shape memory effect is typically initiated by a change in temperature and has been observed in metals, ceramics, and polymers. From a macroscopic point of view, the shape memory effect in polymers differs from ceramics and metals due to the lower stresses and larger recoverable strains achieved in polymers.

Several existing devices have incorporated shape memory metals into a hernia patch. For example, in U.S. Pat. No. 6,669,735, a combination of synthetic mesh is supported on a ring of shape memory metal alloy for use as a hernia repair patch. Similarly, another hernia repair patch is described in U.S. Patent Application Publication No. 2007/0265710 that uses a shape memory alloy (i.e., Nitinol) or shape memory polymer (Polynorbornene) as a frame for the synthetic mesh of the patch.

The information included in this Background section of the specification, including any references cited herein and any description or discussion thereof, is included for technical reference purposes only and is not to be regarded subject matter by which the scope of the invention is to be bound.

### SUMMARY

Disclosed herein are shape memory polymer (SMP) integrated fabrics that may be used for a variety of medical applications. For example, the SMP-integrated fabrics disclosed herein may be used in a hernia repair patch. Numerous formulations of SMP are integrated with several existing clinically available medical fabrics including, for example: polypropylene mesh, polytetrafluoroethylene (PTFE or GoreTex®), and Dacron®, giving these existing materials unique properties that address several unmet clinical needs. The SMP portion of a SMP-integrated fabric can be fabricated in varying thicknesses with the minimum thickness determined by the thickness of the underlying fabric and up to almost any thickness.

In one implementation, a shape memory polymer integrated medical fabric for use in a surgical procedure is disclosed. The integrated fabric includes a medical fabric and a shape memory polymer integrated with the medical fabric to provide a deformable and reformable structure to the integrated fabric upon placement in vivo. The shape memory polymer may be integrated with the medical fabric in a pattern that leaves portions of the medical fabric uncoated. The surgical procedure may be repair of a hernia and the integrated medical fabric is a hernia repair patch. The shape memory polymer may include thiol and/or vinyl monomers or oligomers. The shape memory polymer may further include acrylate or methacrylate functional groups. The shape memory polymer may be a 10 wt % PEGDMA with a Mₙ=1000 and remainder tert-butyl acrylate with 0.1 wt % photoinitiator (2,2 dimethoxy-2-phenylacetopenone).

In another implementation, a method of forming a shape memory polymer-integrated fabric is disclosed. The method may include providing a medical fabric and placing the medical fabric in a mold gasket. A shape memory polymer of a desired formulation is applied to a surface of the medical fabric and a pair of transparent slides are placed on each side of the mold gasket to retain the shape memory polymer against the medical fabric. The shape memory polymer is exposed to ultraviolet light to cure the shape memory polymer. The integrated medical fabric with the cured shape memory polymer is then removed or released from the mold gasket. In some implementations, a mask may be placed on or a wax layer may be placed adjacent to the medical fabric before applying the shape memory polymer to prevent the shape memory polymer from integrating with certain portions of the medical fabric covered by the mask or adjacent to the wax. The method may also include sterilization of the SMP-integrated fabric by steam or chemical sterilization.

In another implementation, a molding apparatus for forming a shape memory polymer-integrated medical fabric is disclosed herein. The apparatus includes at least two molding gaskets operably attached to opposing sides of a medical fabric and a pair of transparent slides in retaining engagement with a shape memory polymer disposed about the medical fabric. The apparatus may further include an ultraviolet light source configured to cure the shape memory polymer disposed about the medical fabric to create a shape memory polymer integrated medical fabric.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention is provided in the following written description of various embodiments of the invention, illustrated in the accompanying drawings, and defined in the appended claims.

### BRIEF DESCRIPTION OF THE DRAWINGS

FIG. 1A is a schematic cross-section view of an exemplary implementation of a mold set-up for curing a SMP-integrated fabric.

FIG. 1B is a schematic top plan view of a SMP-integrated fabric formed by the mold set-up of FIG. 1A.

FIG. 1C is a schematic side elevation view of a SMP-integrated fabric formed by the mold set-up of FIG. 1A.

FIG. 1D is a schematic enlarged, cross-section view of a strand of SMP-integrated fabric formed by the mold set-up of FIG. 1A.

FIG. 2A is a schematic cross-section view of another exemplary implementation of a mold set-up for curing a patterned SMP-integrated fabric.

FIG. 2B is a schematic top plan view of a patterned SMP-integrated fabric formed by the mold set-up of FIG. 2A.

FIG. 3A is a schematic top plan view of another exemplary implementation of a patterned SMP-integrated fabric with SMP-integrated lateral edges.

FIG. 3B is a schematic side elevation view of the SMP-integrated fabric of FIG. 3A with SMP-integrated lateral edges.

FIG. 4 is a schematic top plan view of another exemplary implementation of a patterned SMP-integrated fabric with SMP-integrated borders.

FIG. 5A is a schematic cross-section view of another exemplary implementation of a mold set-up for curing a one-sided SMP-integrated fabric.

FIG. 5B is a schematic side elevation view of the one-sided SMP-integrated fabric of FIG. 5A.

FIG. 6A is a schematic top plan view of another exemplary implementation of a patterned SMP-integrated fabric with offset SMP-integrated borders.

FIG. 6B is a schematic top plan view of another exemplary implementation of a patterned SMP-integrated fabric.

FIG. 7 is a flow diagram of an exemplary process for manufacturing a SMP-integrated fabric.

FIG. 8 is a flow diagram of an exemplary process for manufacturing a woven SMP fabric.

FIG. 9 is a flow diagram of an exemplary process for manufacturing a woven SMP-integrated fabric.

### DETAILED DESCRIPTION

As disclosed herein, shape memory polymer (SMP)-integrated fabrics may have a large variety of patterns based upon how the shape memory polymer is integrated into the base fabric. For example, in one exemplary implementation, the shape memory polymer may be integrated along the edges of the fabric while the center of the fabric remains free of SMP. In a medical application, such a pattern may facilitate integration with surrounding tissue while still maintaining shape memory functionality. In another embodiment, the SMP border may be offset inwards a few millimeters to provide a fabric only border for suturing the SMP-integrated fabric to surrounding tissue. In alternate embodiments, the shape memory polymer may be integrated with the fabric in a variety of patterns of coated and uncoated areas. Further, shape memory polymer may be integrated with fabrics in ways that maintain the material thickness of the original fabric.

Integration of the SMP with the traditional fabrics does not alter the shape memory functionality of the SMP. The indicates that all of the design tools for controlling activation rate for traditional SMP materials apply to SMP-integrated fabrics. SMP-integrated fabrics may also be steam sterilized without loss of shape memory functionality. Other sterilization techniques are also possible.

By using multiple formulations of shape memory polymer on a single piece of fabric, a large combination of material properties may be provided within a single SMP-integrated fabric device. For example, the shape memory polymer integrated with the superior and inferior edges of a piece of fabric may be formulated to have a relatively high modulus value to create a stiff, structurally supporting section, while the lateral portions of the fabric may be coated with a shape memory polymer having lower modulus values to provide sections that conform better to the surrounding tissue.

In certain exemplary implementations, acrylate and thiol based shape memory polymers are used in a hernia repair patch. These shape memory polymers are advantageous over existing hernia patches because of the flexibility that exists in customizing the material properties and the shape memory effects, for example, variable stiffness and activation time. The material disclosed herein improves upon prior hernia patch designs by using a customizable shape memory polymer as opposed to a rigid shape memory metal alloy or non-customizable polymer. The SMP hernia patch may also have differing material properties within the device so that one section may be soft and self-conforming to the surrounding tissue and another section may be rigid to provide structural support. In addition, the shape memory polymer formulations may be tailored so that the temperature of activation and the rate of activation can be varied based on application requirements.

In one embodiment, the shape memory material is incorporated throughout the hernia patch. In another embodiment, shape memory polymer is integrated with the hernia patch in various patterns, for example, a cross-hatch, circles and other curved shapes, rectangles or other polygonal shapes, or lines. These patterns provide better conformance between the SMP hernia patch and tissue. By varying the amounts of shape memory polymer and surgical mesh material (e.g., Dacron®, polypropylene, or Goretex®), varying levels of tissue incorporation and/or adhesion with the SMP hernia repair patch may be achieved. For example, the surgical mesh material may be completely encapsulated in shape memory polymer, which would inhibit mesh integration into tissue. Alternatively, only one side of the mesh material or only a section of the mesh material may be coated with the shape memory polymer, which would allow the remaining mesh to be absorbed into the tissue. Further, the porosity of the shape memory polymer material may be varied to thereby allow even greater amounts of mesh encapsulation within tissue.

Shape Memory Polymers

Basic thermomechanical response of shape memory polymer (SMP) materials is defined by four critical temperatures. The glass transition temperature, T_{g}, is typically represented by a transition in modulus-temperature space and can be used as a reference point to normalize temperature. SMPs offer the ability to vary T_{g} over a temperature range of several hundred degrees by control of chemistry or structure. The predeformation temperature, T_{d}, is the temperature at which the polymer is deformed into its temporary shape. Depending on the required stress and strain level, the initial deformation at T_{d} can occur above or below T_{g}. The storage temperature, Tₛ, represents the temperature in which no shape recovery occurs and is equal to or below T_{d}. At the recovery temperature, Tᵣ, the shape memory effect is activated, which causes the material to recover its original shape, and is typically in the vicinity of T_{g}. Recovery can be accomplished isothermally by heating to a fixed Tᵣ and then holding, or by continued heating up to and past Tᵣ. From a macroscopic viewpoint, a polymer will demonstrate a useful shape memory effect if it possesses a distinct and significant glass transition and a large difference between the maximum achievable strain, εₘₐₓ, during deformation and permanent plastic strain after recovery, εₚ. The difference εₘₐₓ−εₚ is defined as the recoverable strain, ε_{recover}, while the recovery ratio is defined as ε_{recover}/εₘₐₓ.

The microscopic mechanism responsible for shape memory in polymers depends on both chemistry and structure. The primary driving force for shape recovery in polymers is the low conformational entropy state created and subsequently frozen during the thermomechanical cycle. If the polymer is deformed into its temporary shape at a temperature below T_{g}, or at a temperature where some of the hard polymer regions are below T_{g}, then internal energy restoring forces will also contribute to shape recovery. In either case, to achieve shape memory properties, the polymer must have some degree of chemical crosslinking to form a “memorable” network or must contain a finite fraction of hard regions serving as physical crosslinks.

Shape memory polymer materials may be used for a wide variety of applications. Their ability to recover strains imparted upon them, in a manner that is different than pure thermal expansion, due to an external stimulus, makes SMP materials well suited for many applications, such as biological and general mechanical. The external stimulus that activates SMPs may be heat, light, or other stimuli known to those having skill in the art. SMPs which use heat as an external stimulus often have temperatures at which transition occurs.

A transition temperature can be a property of a material (e.g., SMP, thermoplastic, thermoset). A transition temperature may be defined through a number of methods/measurements and different embodiments may use any of these different methods/measurements. For example, a transition temperature may be defined by a temperature of a material at the onset of a transition (Tₒₙₛₑₜ), the midpoint of a transition, or the completion of a transition. As another example, a transition temperature may be defined by a temperature of a material at which there is a peak in the ratio of a real modulus and an imaginary modulus of a material (e.g., peak tan-δ). It should be noted that the method of measuring the transition temperature of a material may vary, as may the definition of steps taken to measure the transition temperature (e.g., there may be other definitions of tan-δ).

A transition temperature may be related to a number of processes or properties. For example, a transition temperature may relate to a transition from a stiff (e.g., glassy) behavior to a rubbery behavior of a material. As another example, a transition temperature may relate to a melting of soft segments of a material. A transition temperature may be represented by a glass transition temperature (T_{g}), a melting point, or another temperature related to a change in a process in a material or another property of a material.

In addition, molecular and/or microscopic processes, including those processes around a transition temperature, may be related to the macroscopic properties of the material. From a macroscopic viewpoint, as embodied in a modulus-temperature graph, a polymer's shape memory effect may possess a glass transition region, a modulus-temperature plateau in the rubbery state. A polymer's shape memory effect may include, as embodied in a stress-strain graph, a difference between the maximum achievable strain, εₘₐₓ, during deformation and permanent plastic strain after recovery, εₚ. The difference εₘₐₓ−εₚ may be considered the recoverable strain, ε_{recover}, while the recovery ratio (or recovery percentage) may be considered ε_{recover}/εₘₐₓ.

The properties of SMPs can be controlled by changing the formulation of the SMP, or by changing the treatment of the SMP through polymerization and/or handling after polymerization. The techniques of controlling SMP properties rely on an understanding of how SMP properties are affected by these changes and how some of these changes may affect more than one property. For example, changing the percentage weight of a cross-linker in a SMP formulation may change both a transition temperature of the SMP and a modulus of the SMP. In one embodiment, changing the percentage weight of a cross-linker will affect the glass transition temperature and the rubbery modulus of an SMP. In another embodiment, changing the percentage weight of cross-linker will affect a recovery time characteristic of the SMP.

Some properties of a SMP may be interrelated such that controlling one property has a strong or determinative effect on another property, given certain assumed parameters. For example, the force exerted by a SMP against a constraint after the SMP has been activated may be changed through control of the rubbery modulus of the SMP. Several factors, including a level of residual strain in the SMP enforced by the constraint, will dictate the stress applied by the SMP, based on the modulus of the SMP. The stress applied by the SMP is related to the force exerted on the constraint by known relationships.

Examples of constituent parts of the SMP formulation include monomers, multi-functional monomers, cross-linkers, initiators (e.g., photo-initiators), and dissolving materials (e.g., drugs, salts). Two commonly included constituent parts are a linear chain and a cross-linker, each of which are common organic compounds such as monomers, multi-functional monomers, and polymers.

A cross-linker (or “crosslinker”), as used herein, may mean any compound comprising two or more functional groups (e.g., acrylate, methacrylate), such as any poly-functional monomer. For example, a multi-functional monomer is a poly ethylene glycol (PEG) molecule comprising at least two functional groups, such as di-methacrylate (DMA), or the combined molecule of PEGDMA. The percentage weight of cross-linker indicates the amount of the poly-functional monomers placed in the mixture prior to polymerization (e.g., as a function of weight), and not necessarily any direct physical indication of the as-polymerized “crosslink density.”

Because SMP material requires both a thermal transition and a form of crosslinking to possess shape-memory characteristics, the polymer is typically synthesized from a linear chain building mono-functional monomer (tert-butyl acrylate) and a crosslinking di-functional monomer (poly (ethylene glycol) dimethacrylate). Because the crosslinking monomer has two methacrylate groups, one at each end, it is possible to connect the linear chains together. This linear monomer portion can be used to help control the glass transition temperature of the network as well as its overall tendency to interact with water. Thus, the linear portion of the network remains an important and tailor-able portion of the composition.

A linear chain may be selected based on a requirement of a particular application, because of the ranges of rubbery moduli and recovery forces achieved by various compositions. In one embodiment, a SMP with a high recovery force and rubbery modulus may be made from a formulation with methyl-methacrylate (MMA) as the linear chain. In another embodiment, a SMP with a lower recovery force and rubbery modulus may be made from a formulation with tert-butyl acrylate (tBA) as the linear chain. In other embodiments, other linear chains may be selected based on desired properties such as recovery force and rubbery modulus.

In one embodiment, the copolymer network consists of two acrylate-based monomers. In one example of this embodiment, tert-butyl acrylate may be crosslinked with poly (ethylene glycol), dimethacrylate (PEGDMA) via photopolymerization to form a cross-linked network. One subset of this formulation may consist of 10 wt % PEGDMA with a Mₙ=1000 and remainder tert-butyl acrylate with 0.1 wt % photoinitiator (2,2 dimethoxy-2-phenylacetopenone). This exemplary polymer network has a glass transition temperature T_{g} of about 45° C., which offers shape memory activation along with a reasonably soft compliance at body temperature. Furthermore, it has a low rubbery modulus of approximately 1-2 MPa, which is indicative of a low degree of crosslinking that allows for greater packaging deformations and higher strains to failure. In some embodiments, the molecular weight of the PEGDMA may be varied to control hydrophobicity and/or hydrophylicity. This may allow better integration with medical fabrics or meshes that are generally more hydrophobic or hydrophilic. In some embodiments, where a stiffer medical fabric is used, the PEGDMA:tBA wt percentage is increased such that the SMP has sufficient stored force to allow deployment of the SMP integrated material. In still other embodiments, addition of thiol groups may allow better control of manufacturing for composite systems since oxygen inhibition could be decreased leading to better polymerization.

The SMP material may be further varied to enhance desired properties. The SMP material may be photopolymerized from several different monomers and/or homopolymers to achieve a range of desired thermomechanical properties. A SMP formed from three or more monomers and/or homopolymers may achieve a range of rubbery modulus to glass transition temperatures, rather than a strictly linear relationship between these two thermomechanical properties. For example, tert-butyl acrylate may be substituted by 2-hydroxyethyl methacrylate or methyl methylacrylate to create either more hydrophilic or stronger networks, if desired. Additionally, if a hydrophilic monomer such as 2-hydroxyethyl methacrylate is substituted for tert-butyl acrylate, the SMP has the ability to swell post-implantation through hydrogel mechanisms.

Representative natural polymer blocks or polymers include proteins such as zein, modified zein, casein, gelatin, gluten, serum albumin, and collagen, and polysaccharides such as alginate, celluloses, dextrans, pullulane, and polyhyaluronic acid, as well as chitin, poly(3-hydroxyalkanoate)s, especially poly(.beta.-hydroxybutyrate), poly(3-hydroxyoctanoate) and poly(3-hydroxyfatty acids). Representative natural biodegradable polymer blocks or polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers.

Representative synthetic polymer blocks or polymers include polyphosphazenes, poly(vinyl alcohols), polyamides, polyester amides, poly(amino acid)s, synthetic poly(amino acids), polyanhydrides, polycarbonates, polyacrylates, polyalkylenes, polyacrylamides, polyalkylene glycols, polyalkylene oxides, polyalkylene terephthalates, polyortho esters, polyvinyl ethers, polyvinyl esters, polyvinyl halides, polyvinylpyrrolidone, polyesters, polylactides, polyglycolides, polysiloxanes, polyurethanes and copolymers thereof. Examples of polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate).

Synthetically modified natural polymers include cellulose derivatives such as alkyl celluloses, hydroxyalkyl celluloses, cellulose ethers, cellulose esters, nitrocelluloses, and chitosan. Examples of cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt. These are collectively referred to herein as “celluloses”.

Representative synthetic degradable polymer segments include polyhydroxy acids, such as polylactides, polyglycolides and copolymers thereof; poly(ethylene terephthalate); polyanhydrides, poly(hydroxybutyric acid); poly(hydroxyvaleric acid); poly[lactide-co-(.epsilon.-caprolactone)]; poly[glycolide-co-(.epsilon.-caprolactone)]; polycarbonates, poly(pseudo amino acids); poly(amino acids); poly(hydroxyalkanoate)s; polyanhydrides; polyortho esters; and blends and copolymers thereof. Polymers containing labile bonds, such as polyanhydrides and polyesters, are well known for their hydrolytic reactivity. Hydrolytic degradation rates of these polymers may be altered by simple changes in the polymer backbone and the polymer's sequence structure.

Examples of non-biodegradable synthetic polymer segments include ethylene vinyl acetate, poly(meth)acrylic acid, polyamides, polyethylene, polypropylene, polystyrene, polyvinyl chloride, polyvinylphenol, and copolymers and mixtures thereof.

Hydrogels can be formed from polyethylene glycol, polyethylene oxide, polyvinyl alcohol, polyvinyl pyrrolidone, polyacrylates, poly (ethylene terephthalate), poly(vinyl acetate), and copolymers and blends thereof.

The polymers can be obtained from commercial sources such as Sigma Chemical Co., St. Louis, Mo.; Polysciences, Warrenton, Pa.; Aldrich Chemical Co., Milwaukee, Wis.; Fluka, Ronkonkoma, N.Y.; and BioRad, Richmond, Calif. Alternately, the polymers can be synthesized from monomers obtained from commercial sources.

Various SMP properties may be controlled via variations in a cross-linker in the SMP formulation. A range of average molecular weights of cross-linker material for use in a SMP may be determined based upon the desired transition temperature, for example, a transition temperature close to human body temperature. The transition temperature affects the range of possible average molecular weights of cross-linker material that may be used in the SMP because certain combinations of average molecular weights and of percentage weights of cross-linker produce certain transition temperatures and other combinations produce other transition temperatures.

A range of percentage weights of cross-linker material for use in a SMP is also determined from the selected transition temperature. Certain combinations of average molecular weights of cross-linker and percentage weights of cross-linker may be used in the SMP formulation to achieve a certain transition temperature. Determining the range of percentage weight cross-linker and the range of molecular weights may be performed based upon a relationship between transition temperature, molecular weight, and percentage weight cross-linker. The relationship is specific to the linear chain and cross-linker used. Other inputs or manufacturing techniques may also affect the relationship and eventual transition temperature of a SMP.

In one embodiment, empirically-derived relationships which relate molecular weight and weight percentage cross-linker to (a) the transition temperature, (b) the rubbery modulus, and/or (c) a recovery time characteristic may be used. The range of rubbery moduli is determined by evaluating the relationship between rubbery modulus, percentage weight of cross-linker, and molecular weights for a number of combinations determined. This results in a range of possible rubbery moduli for SMPs that also has the desired transition temperature. In another embodiment, relationships may be derived from known theoretical models.

A rubbery modulus is selected from a range of rubbery moduli of as an initial goal value of rubbery modulus for the SMP. The modulus selection may alternatively be performed after a transition temperature is selected, which produces another range of rubbery moduli. In other words, the method may be performed iteratively, repeatedly, and/or in parts. The molecular weight and percentage weight of cross-linker is determined based on the selected rubbery modulus by using the relationship between rubbery modulus, molecular weight and percentage weight of cross-linker to find the combination of molecular weight and percentage weight that corresponds to the rubbery modulus selected.

In another embodiment, determining a range of molecular weights and percentage weights of cross-linker may be performed by creating and/or selecting a table, graph, or chart corresponding to a desired transition temperature or a desired rubbery modulus among a plurality of tables, graphs, and/or charts. In this embodiment, the tables, graphs, and/or charts include information from the relationships described above and outline ranges of molecular weights and percentage weights cross-linker that correspond to the desired value of the property (e.g., transition temperature).

In some implementations, the shape memory polymer may comprise thiol and/or vinyl monomers or oligomers. In some implementations, monomers or oligomers with acrylate or methacrylate functional groups may be combined with thiol and/or vinyl monomers or oligomers.

A thiol-vinyl SMP system includes molecules containing one or more thiol functional groups, which terminate with —SH, and molecules containing one or more vinyl functional groups, which contain one or more carbon-carbon double bonds. The vinyl functional groups in the system may be provided by, for example, allyl ethers, vinyl ethers, norborenes, acrylates, methacrylates, acrylamides or other monomers containing vinyl groups. In some implementations, additional fillers, molecules, and functional groups may be provided to tailor and provide additional properties. In different embodiments, the thiol-ene system has about 1-90% of its functional groups as thiol functional groups or 2%-65% thiol functional groups. The balance of the functional groups (35% to 98% of the functional groups may be vinyl functional groups. In an embodiment, 5-60 mol % of the functional groups in the system may be thiol functional groups and 95-40 mol % vinyl functional groups. In the present invention, the system of molecules containing thiol functional groups and the molecules forming vinyl functional groups is capable of forming a network.

In one class of thiol-vinyl systems, the vinyl monomer is not readily homopolymerizable and is termed an ene monomer. In these systems, the polymerization proceeds via a radically initiated step growth reaction between multifunctional thiol and ene monomers. The reaction proceeds sequentially, via propagation of a thiyl radical through a vinyl functional group. This reaction is followed by a chain transfer of a hydrogen radical from the thiol which regenerates the thiyl radical. the process then cycles many times for each radical generated in the photoinitiation step. This successive propagation/chain transfer mechanism is the basis for thiol-ene polymerization.

Thiol bearing monomers suitable for implementations of thiol-vinyl shape memory polymer systems include any monomer or oligomer having thiol (mercapatan or “SH”) functional groups. Thiols are any of various organic compounds or inorganic compounds having the general formula RSH which are analogous to alcohols but in which sulfur replaces the oxygen of the hydroxyl group. Suitable monomers or oligomers may have one or more functional thiol groups. In an embodiment, the monomer or oligomer cannot be considered a polymer in its own right. In different embodiments, the monomer or oligomer has an average molecular weight less than 10,000, less than 5,000, less than 2,500, less than 1000, less than 500, from 200 to 500, from 200-1000, from 200-1,500, from 200-2000, from 200-2,500, from 200-5000, or from 200-10,000. In different embodiments, the monomer or oligomer has at least two thiol functional groups, at least three thiol functional groups, at least four thiol functional groups, at least five thiol functional groups, at least six thiol functional groups or from 2 to 4 thiol functional groups. Examples of suitable thiol bearing monomers include: pentaerythritol tetra(3-mercaptopropionate) (PETMP); trimethylolpropane tris(3-mercaptopropionate) (TMPTMP); glycol dimercaptopropionate (GDMP); IPDU6Th; and 1,6-hexanedithiol (HDTT), and benzene diol.

Monomers or oligomers having vinyl functional groups suitable for implementations of thiol-vinyl shape memory polymer systems include any monomer or oligomer having one or more functional vinyl groups, i.e., reaching “C═O” groups. In an embodiment, the monomer or oligomer cannot be considered a polymer in its own right. In different embodiments, the monomer or oligomer has an average molecular weight less than 10,000, less than 5,000, less than 2,500, less than 1000, less than 500, from 200 to 500, from 200-1000, from 200-1,500, from 200-2000, from 200-2,500, from 200-5000, or from 200-10,000. In different embodiments, the monomer or oligomer has at least two vinyl functional groups, at least three vinyl functional groups, at least four vinyl functional groups, at least five vinyl functional groups, at least six vinyl functional groups, or from 2 to 4 vinyl functional groups. Examples of suitable vinyl monomers include: allyl pentaerythritol (APE); triallyl triazine trione (TATATO); trimethylolopropane diallyl ether (TMPDAE); hexanediol diacrylate (HDDA); trimethylolpropane triacrylate (TMPA); Ebecryl 8402; Vectomer 5015; and IPDU6AE.

Monomers or oligomers with acrylate or methacrylate functional groups may also be combined with thiol and/or vinyl monomers or oligomers using, as one example, a chain transfer agent process with the agent being thiol. Exemplary acrylate and methacrylate monomers for use with thiol-vinyl shape memory polymer systems include tricyclodecane dimethanol diacrylate; tricyclodecane dimethanol dimethacrylate; bisphenol-A ethoxylated diacrylate; bisphenol-A ethoxylated dimethacrylate; bisphenol-A epoxy diacrylate; bisphenol-A epoxy dimethacrylate; urethane acrylates; urethane methacrylates; polyethylene glycol diacrylate; polyethylene glycol dimethacrylate and commercial monomers. Commercial monomers include aliphatic urethane acrylates such as Ebecryl 8402; Ebecryl 230; Loctite 3494; Ebecryl 4833; Ebecryl 3708.

The monomer or oligomer comprising a vinyl group may further comprise at least one urethane group. In an embodiment, the monomer comprises from 2-4 or 2-6 urethane groups. In an embodiment, the oligomer comprises from 4-40 urethane groups. A monomer comprising urethane groups may be formed by reacting a polyisocyanate with a molecule comprising an alcohol group and at least two vinyl groups. For example, a diisocyanate could be reacted with a trimethylolpropane diallyl ether or allyl pentaerythritol.

Thiol-vinyl systems for shape memory polymers may also include and/or utilize various initiators, fillers, and accelerators, depending on the application. For example, if photopolymerization using visible light is desired, a commercially available photoinitiator such as Irgacure 819 or Irgacure 784 (manufactured by Ciba Specialty Chemicals Co. (http://www.cibasc.com)) may be used. If ultraviolet photopolymerization is desired, then 2,2-dimethyloxy-2-pheynlacetophenone (Irgacure 651, Ciba Specialty Chemicals Co.) may be used as an initiator or 1-hydroxy-cyclohexyl-phenyl-ketone (Irgacure 184, Ciba Specialty Chemicals).

A thiol-yne system includes molecules containing one or more thiol functional groups, which terminate with —SH, and molecules containing one or more yne functional groups, which contain one or more carbon-carbon triple bonds. The functional groups in the system may be provided by, octadiyne or heptadiyne for example, or other monomers containing yne groups.

SMP-Integrated Fabrics

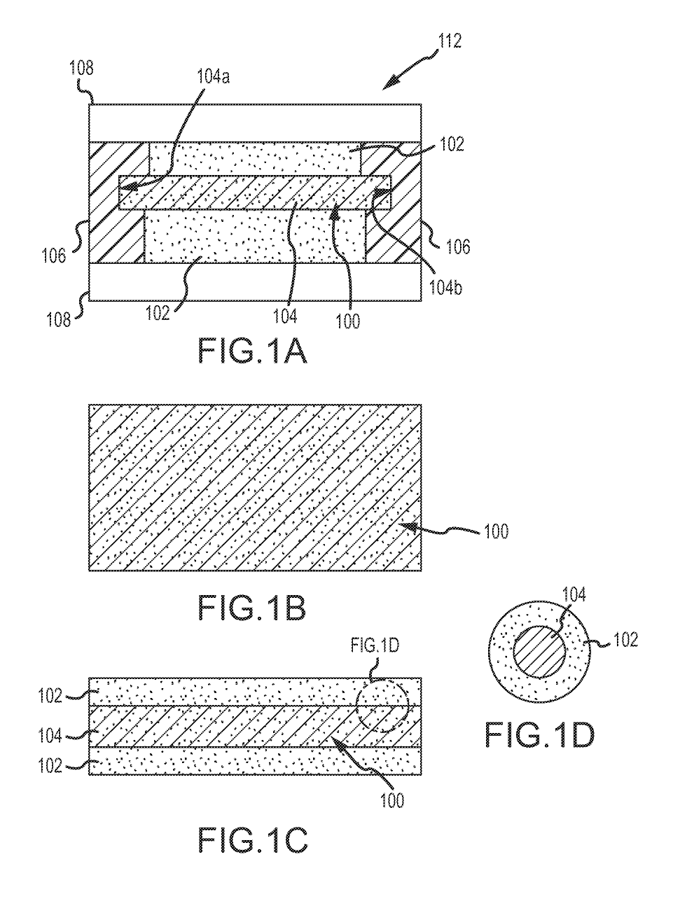

For a discussion of medical fabrics that may utilize a SMP as discussed above, reference is now made to FIGS. 1A-5B which illustrate various embodiments of a SMP-integrated fabric and apparatus for making the same.

FIGS. 1A-1D depict a first exemplary implementation of a SMP-integrated fabric 100 and an exemplary apparatus for forming the same. As depicted in FIG. 1A, a medical fabric 104 is held at its ends 104a,104b with a molding gasket 106 and is further coated on each side with a shape memory polymer 102. Because the medical fabric 104 is formed by the interconnection of woven fibers, the medical fabric 104 is by nature porous, thus allowing the shape memory polymer 102 to integrate with the medical fabric 104 and coat each individual thread or strand making up the medical fabric 104. Such coating is depicted in FIG. 1D where the shape memory polymer 102 is depicted as completely surrounding an individual strand of the medical fabric 104. Returning to FIG. 1A, a plate of glass 108 (e.g., a glass slide) may be placed on each side of the coated medical fabric 104 and supported and separated by the molding gaskets 106. The glass plate 108 retains the shape memory polymer 102 about the medical fabric 104 in a thin layer for the curing process. The molding apparatus 112 (e.g. the glass 108, the molding gaskets 106 and the material found therebetween (e.g. the SMP 102 disposed about the medical fabric 104)) is then exposed to ultraviolet light which passes through each of the glass plates to cure the shape memory polymer 102, thus binding it around the strands of the medical fabric 104 to create the SMP-integrated fabric 100.

As shown in FIGS. 1D and 1C, the shape memory polymer 102 both presents as a coating on each side of the medical fabric 104 and integrates through the weave of the medical fabric 104, for example, by fully coating each individual thread or strand of the medical fabric 104. Depending on the thickness of the threads of strands that make up the medical fabric 104 and the tightness of the weave of the medical fabric 104, the nature of the integration of the shape memory polymer 102 with the medical fabric 104 may differ between the materials. For example, more tightly woven materials with a smaller pore between strands of the medical fabric 104 may limit the ability of the shape memory polymer 102 to fully coat the individual strands but instead present more as a top coating on each side of the medical fabric. Alternatively, medical fabric 104 that is less tightly woven allows the shape memory polymer 102 to completely coat (or almost completely coat) the individual strands of the medical fabric 104 rather than merely a macro coating on the surfaces of the medical fabric 104.

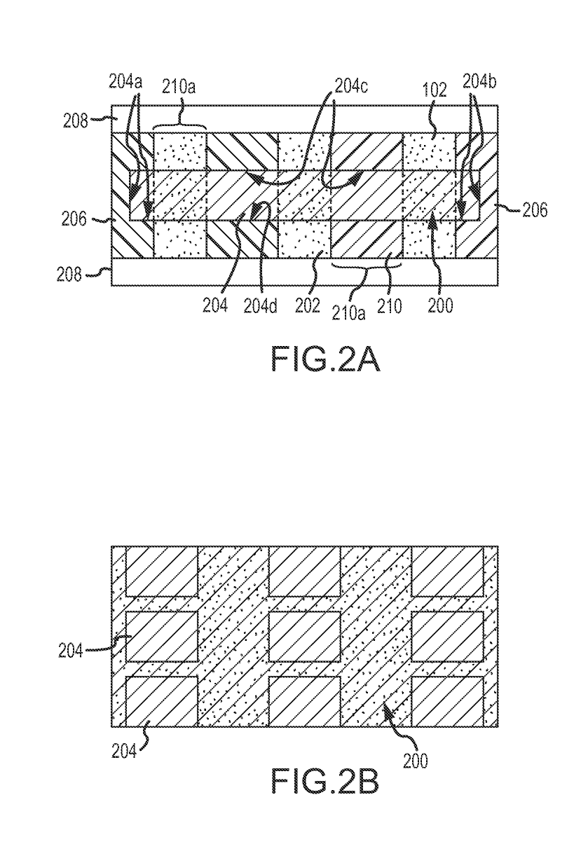

FIGS. 2A and 2B show an alternative implementation of a SMP-integrated fabric 200. The mold apparatus is depicted in FIG. 2A with the medical fabric 204 held at its edges 204a, 204b between molding gaskets 206. In this implementation, in addition to the molding gaskets 206 on the edges 204a, 204b, a mask 210 is formed on each side 204c, 204d of the medical fabric 204. The mask 210 may be made of the same material as the molding gasket 206 and may be an integrated structure with the molding gasket 206. The mask 210 may also be formed in any desired pattern to achieve a desired effect in the SMP-integrated fabric 200. In this implementation, the mask 210 is formed in a grid pattern leaving rectangular openings 210a. Other grid patterns with different geometrically shaped openings are possible, for example, circles or other curved shapes; squares, triangles, or other polygonal shapes; or a combination of any of these patterns. In the implementation shown in FIG. 2A, a mask 210 is placed on both sides of the medical fabric 204. However, in certain implementations it may be desirable to mask only one side of the medical fabric 204. The masked medical fabric 204 may then be coated with shape memory polymer 202 which fills the void areas 210a within the mask and coats the exposed portions of the medical fabric 204 within the openings in the mask 210. A pair of glass plates or slides 208 is placed on each side of the molding gaskets 206 and the mask 210 to retain the shape memory polymer 202 and the completed mold (i.e. molding apparatus 212) is exposed to ultraviolet light to cure the shape memory polymer material 202 to complete the integration process. The completed SMP-integrated fabric 200 is shown in FIG. 2B having a pattern of areas integrated with the shape memory polymer 202 and other areas of uncoated medical fabric 204.

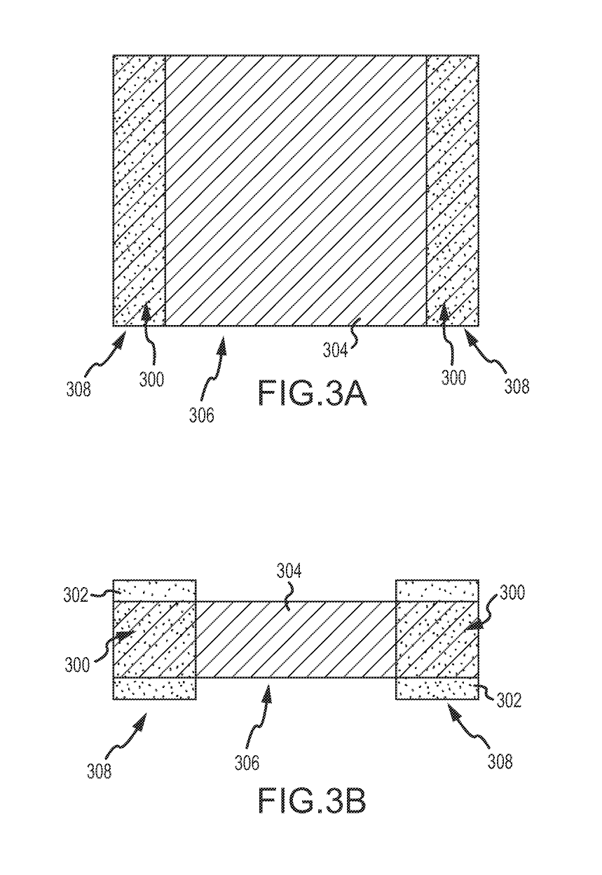

A further implementation of a SMP-integrated fabric 300 is shown in FIGS. 3A and 3B. In this implementation, the medical fabric 304 is only coated in strips along two opposing edges of the medical fabric 304. As shown in FIG. 3B, the medical fabric is uncoated in the middle 306 while two opposing edges 308 are coated with shape memory polymer 302 to form side or edge strips 308 of SMP-integrated fabric 300. These side strips 308 may be formed by a similar masking process as explained above with respect to FIGS. 2A and 2B. In this implementation, however, a middle slot was masked to prevent coating with the shape memory polymer 302.

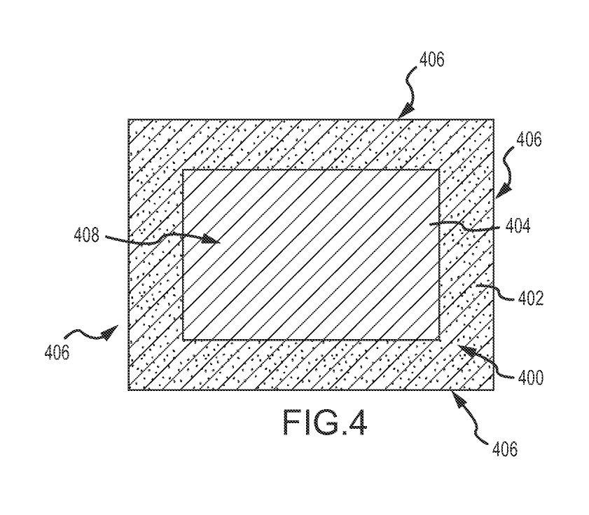

A further implementation of a SMP-integrated fabric 400 is presented in FIG. 4. In this implementation, the medical fabric 404 has each border edge 406 coated with shape memory polymer 402 to result in medical fabric 404 with SMP-integrated fabric 400 completely around the border. The implementation of FIG. 4 can similarly be formed by a masking process as described with respect to FIGS. 2A and 2B by masking a large center area 408 while leaving the borders 406 of the medical fabric 404 exposed for coating with the shape memory polymer 402 and then curing the shape memory polymer 402 to integrate it with the medical fabric 404.

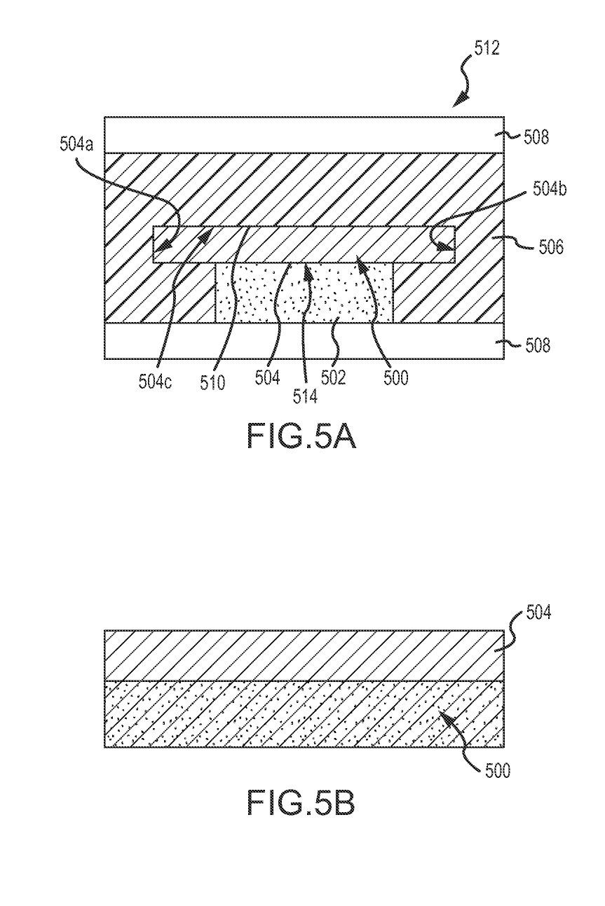

A further implementation of a SMP-integrated fabric 500 is depicted in FIGS. 5A and 5B. In this implementation a one-sided SMP-integrated fabric 500 is formed. The mold setup for this implementation is depicted in FIG. 5A, which shows the medical fabric 504 held within a molding gasket 506. In this implementation, the molding gasket 506 holds the edges 504a, 504b of the medical fabric 504 as in prior embodiments and further extends across one entire side 504c of the medical fabric 504. In addition, the flat side of the medical fabric 504 that is adjacent to the full wall of the molding gasket 506 (i.e. side 504c) is coated with a wax 510 to prevent the shape memory polymer 502 from fully penetrating the medical fabric 504 and entirely coating all the strands. The shape memory polymer 502 is introduced to the exposed side 514 of the medical fabric 504 which it coats and penetrates until it encounters the wax layer 510 on the opposite side of the medical fabric 504. In some implementations, the wax layer may include: dental wax, such as Polysciences Dental Wax (sheets-150×75×1.5 mm) NC960.024.9 or Pink Modeling Dental Wax (11 lbs) 50.948.964, both from Fisher Scientific, Pittsburgh, Pa., USA; bees wax, such as White disc/NF/FCC, Fisher Chemical 8012.89.3, Fisher Scientific, Pittsburgh, Pa., USA; and/or paraffin wax, such as Granular Acros Organics 8002.74.2, Fisher Scientific, Pittsburgh, Pa., USA.

As before, the molding gasket 506 is sandwiched between two glass plates 508 and the entire molding apparatus 512 is subjected to ultraviolet light to cure the shape memory polymer 502. In this instance, the ultraviolet light only impacts the shape memory polymer 502 from one side of the molding apparatus 512. After curing, the glass plates 508 and molding gasket 506 are removed and the wax coating 510 on the back side of the medical fabric 504 is removed with an appropriate solvent that releases the wax 510 from the strands of the medical fabric 504 while not impacting either the medical fabric 504 or the shape memory polymer 502. The resulting product is shown in FIG. 5B in which a one-sided SMP-integrated fabric 500 is formed, wherein the shape memory polymer 502 covers only one face of the medical fabric 504.

In all of the embodiments depicted in FIGS. 1A-5B, once the SMP-integrated fabrics are formed in a desired pattern and configuration, they may be mechanically deformed for storage or for a more suitable configuration for delivery in vivo. For example, the SMP-integrated medical fabrics once formed may be rolled up for delivery through a catheter or a lumen of an endoscope or other instrument. The SMP-integrated medical fabrics may be deployed from the delivery device. Then, upon being subject to an external stimulus, for example, body temperature, the SMP-integrated fabric may unfurl from its rolled configuration and return to its original memory configuration for use by a clinician in a particular procedure for which the SMP-integrated fabric was developed.

In one implementation, SMP-integrated fabrics may be formed as hernia patches. The shape memory polymer portion of a SMP hernia patch may be fabricated in varying thicknesses with the minimum thickness determined by the thickness of the traditional patch fabric (e.g., Dacron®, Gore-Tex®, and polypropylene) and up to almost any thickness. The polypropylene mesh has a coarse weave and is woven of strands of polypropylene. An SMP-integrated polypropylene hernia patch remains quite porous while fully coating all the polypropylene strands with the shape memory polymer. PTFE fabric (e.g. Gore-Tex®) is soft and can be rolled into any shape, but cannot unfurl itself. In contrast, a SMP-integrated PTFE patch can hold the rolled shape of the PTFE fabric and self deploy after the activation time and/or temperature has been reached. Similar performance characteristics may be achieved with the SMP-integrated Dacron® patches. Also, the SMP-integrated Dacron® patch is able to achieve the thinnest patch out of the three clinical patch materials at 0.0135 inches thick. The ability to maintain material thickness of the standard patch fabric, even with the SMP co-polymerization, allows the SMP hernia patch to be used with existing insertion devices. SMP hernia patches may also be steam sterilized without loss of shape memory functionality.

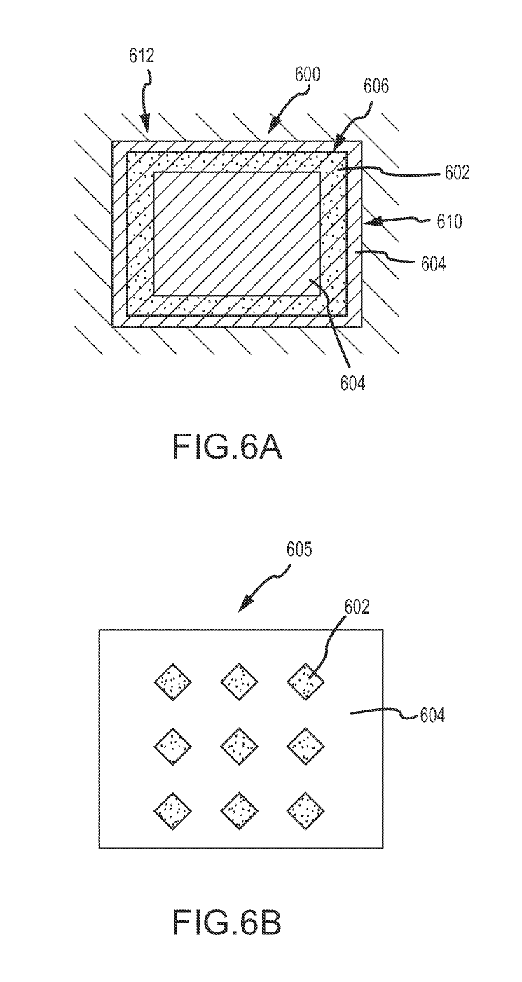

SMP hernia patches may have a large variety of patterns in which the SMP material can be integrated into the patch fabric, including but not limited to those discussed with reference to FIGS. 1A-5B. For example, only the edges may have shape memory polymer while the center of the patch contains no SMP material (see e.g. FIG. 4). This facilitates integration of the fabric portion with surrounding tissue while still maintaining shape memory functionality. In one implementation, as shown in FIG. 6A, the border 606 of SMP material 602 may be offset inwards a few millimeters, for example, to provide a border 610 of the (uncoated) medical fabric 604 for suturing the patch 600 to surrounding tissue 612. In other implementations, the border of SMP material may be offset inwards by greater than or less than a few millimeters. Alternatively, and as shown in FIG. 6B, the center of the SMP hernia patch 605 may contain SMP material 602 laid out in a diamond or other pattern while the edges of the medical fabric 604 have no shape memory polymer coating. In one embodiment, a SMP hernia patch may be constructed in a manner as described above so that the SMP material is only on one side of the patch; the opposite surface side is void of shape memory polymer (see, e.g. FIGS. 5A-5B).

A single SMP hernia patch may have a large combination of material properties. For example, a shape memory polymer coating on the superior and inferior edges may be formulated to have a relatively high modulus value to create a stiff, structural, supporting section, while the lateral portions may be integrated with a shape memory polymer formulation having lower modulus values to provide sections that conform better to the surrounding tissue.

Integration of the SMP with the traditional medical fabrics does not alter the shape memory functionality of the SMP. This indicates that all of the design tools for controlling activation rate for traditional SMP materials apply to SMP hernia patches. A clinically relevant example of this is the ability to control the activation time, or the time that must elapse before the SMP hernia patch will begin to self deploy. For complicated procedures, the activation time may be set to a large value giving the surgeon ample time to place the patch before it self deploys, or for simple surgeries, the activation time may be set low so as to speed up the time to self-deployment.

The shape of the SMP hernia patch has no impact on incorporation of the shape memory polymer or its functionality. That is, the SMP hernia patch is self-deploying, which makes placing the patch easier for the surgeon and reduces surgical time substantially. SMP hernia patches have successfully demonstrated the ability to maintain their packaged shape in repeated fashion. In contrast, commercially available hernia patches exhibit creep, inability to maintain a particular pre-defined shape after deployment, and an inability to deploy on command with the application of thermal energy (e.g., body temperature). SMP hernia patches can also be programmed through proper formulation of the shape memory polymer to activate after precise periods of time have elapsed.

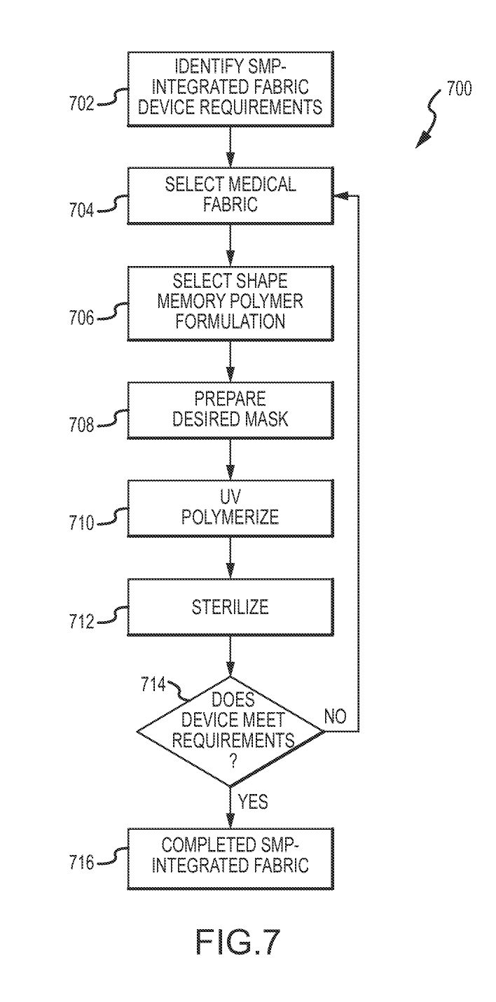

For a discussion of various methods that may be employed for creating SMP-integrated medical fabrics, reference is now made to FIGS. 7-9. FIG. 7 presents an exemplary process for integrating shape memory polymers into fabrics, meshes, patches, and other woven or porous materials that may be used in medical procedures, for example, in surgeries for hernia repairs. The integration process 700 begins at operation 702 in which the particular device requirements are identified by the manufacturer. Exemplary requirements may include the particular application (e.g., ventral hernia repair), desired activation time, desired stiffness, need for tissue integration, and need for suturing. For example, in the case of hernia repair, it may be desirable in particular situations to be able to suture a SMP-integrated medical fabric to surrounding tissue. In such a case it may be desirable to provide sections of uncoated medical fabric in appropriate locations for suturing. In other situations, tissue integration with the medical fabric may be desirable to achieve a better medical result. In such an implementation the pattern of SMP integration may be structured in order to provide appropriate areas of uncoated medical fabric to achieve the desired tissue integration. The particular formulation of the shape memory polymer may also be important in determining factors such as stiffness of the SMP-integrated fabric to achieve the desired medical purpose or the activation time required for the SMP material to return to its original, pre-deformation state to allow sufficient time for delivery or for necessary manipulation by the clinician.

Once the device requirements are identified, the actual medical fabric appropriate to the intended clinical purpose should be selected as indicated in operation 604. For several different types of medical fabric, for example, Gore-Tex®, Dacron®, or polypropylene, may be used depending upon the particular procedure. With respect to these exemplary materials, each provides different benefits. For example, Gore-Tex® has a very tight, close weave and serves as a water barrier. A shape memory polymer coating on Gore-Tex® may have less penetration within the weave. Alternatively, polypropylene generally has a large weave and would remain a porous material even after coating with a shape memory polymer.

Next the actual formula for the desired shape memory polymer is selected as indicated in operation 706. As noted above, the shape memory polymer formulation should be chosen to achieve the desired properties for the SMP-integrated fabric device including, for example, activation time, activation temperature, and rubbery modulus. Such methods of formulation have been previously described herein above and elsewhere as referenced.

Once the medical fabric and shape memory polymer formula have been selected, the manufacturer must then design and implement an appropriate mask in order to achieve a desired shape memory polymer coating pattern on the medical fabric as indicated in operation 708. As discussed above, such patterns may be desirable for providing areas for tissue integration with the medical fabric or for suturing the medical fabric to the patient's tissue.

Once the mask has been determined and the medical fabric has been placed within the appropriate molding gasket and coated with the shape memory polymer, the shape memory polymer may be cured by exposure to ultraviolet light in order to create the deployed or post transition SMP-integrated medical fabric as indicated in operation 710. The SMP-integrated medical fabric may then be sterilized, for example, by exposure to steam or by chemical cleansing as indicated in operation 712. The completed SMP-integrated medical fabric may then be tested as indicated in operation 714 to determine whether the initial device requirements identified in operation 702 are met. If such requirements are met, then the completed SMP-integrated medical fabric may be mechanically deformed for storage and later delivery in a medical operation as indicated in operation 716. Alternatively, if the device requirements set forth in operation 702 have not been met, the manufacturing process 700 may return to step 704 for selection of alternative shape memory polymer formulations or medical device fabrics for integration to form the desired SMP-integrated fabric device.

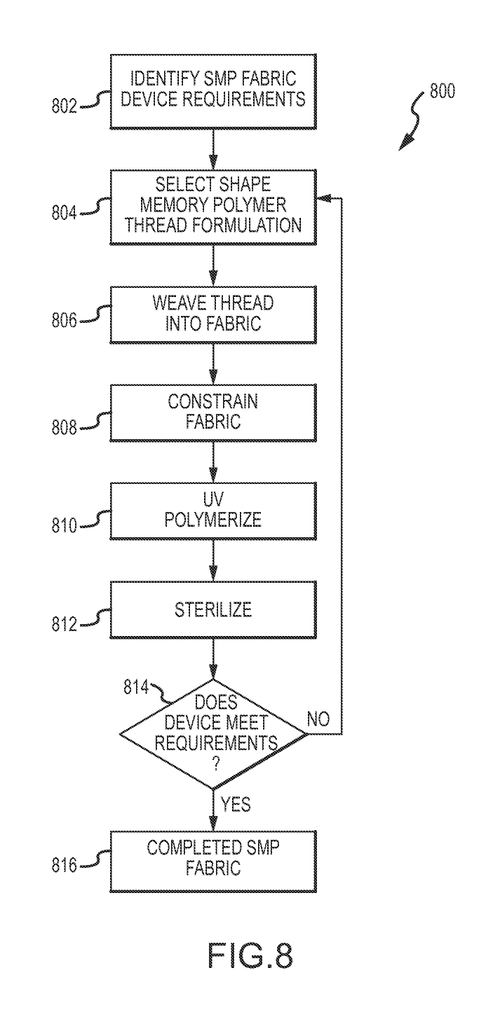

FIG. 8 depicts an alternative process 800 for formation of a SMP medical fabric. In this process 800, a medical fabric is constructed entirely of woven strands shape memory polymer. As in the prior implementation, the first step in the process 800 is to identify the requirements to be placed upon the completed medical fabric device as indicated in operation 802. Next the material properties of the chosen shape memory polymer threads or fibers are selected to meet the device requirements as indicated in operation 804. Again, such material properties may relate to requirements such as activation time, stiffness, or activation temperature. Once the appropriate shape memory polymer formulation is determined, the threads or strands of such shape memory polymer may be woven together to form a fabric as indicated in operation 806. Once woven, the SMP fabric may be placed in a form or otherwise constrained into a desired post-deployment shape as indicated in operation 808. The constrained SMP material may then be cured and thus set in this post deployment shape as indicated in operation 810. The cured SMP fabric may then be sterilized by heat or chemical treatment as previously indicated and as shown in operation 812.

The SMP fabric device thus formed may then be tested as indicated in operation 814 to determine whether the SMP fabric meets the device requirements identified in operation 802. Such testing may include mechanical deformation of the SMP fabric device into an alternative shape suitable for storage or delivery and then subjecting the SMP fabric device to the desired activation temperature to determine whether the SMP fabric device appropriately deploys at the desired temperature and within the desired activation time. The SMP fabric device may be further tested to determine whether its structural rigidity or elasticity is appropriate for the desired medical procedure. Once it is determined that the desired requirements have been met, the SMP fabric device may be mechanically deformed for appropriate storage and delivery in a clinical setting as indicated in operation 816. Alternatively, if the device requirements set forth in operation 802 have not been met, the manufacturing process 800 may return to step 804 for selection of alternative shape memory polymer formulations to form the desired SMP fabric device.

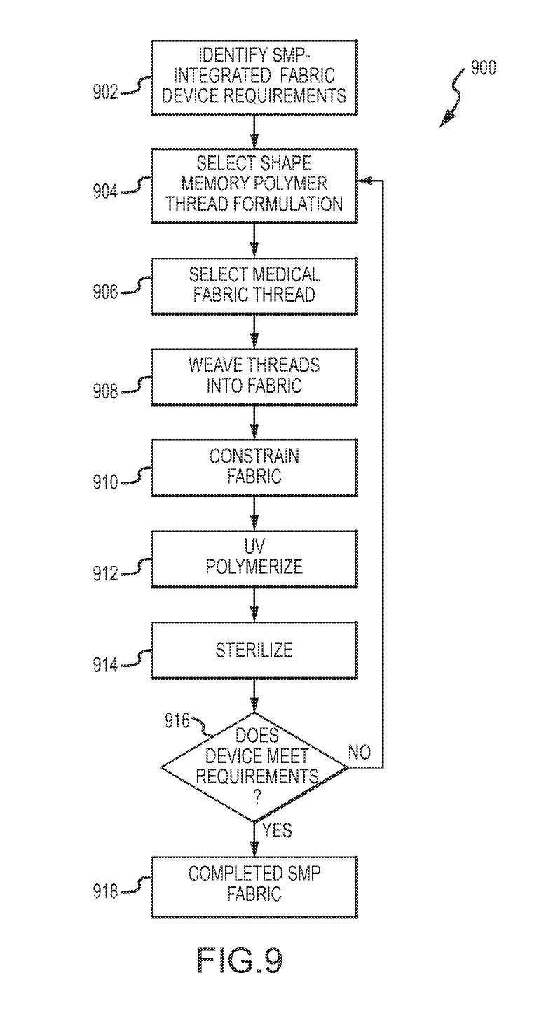

A further process 900 for developing a SMP-integrated fabric is shown in FIG. 9. In this implementation, the process 900 weaves shape memory polymer strands or threads with strands or threads of other fabric material, for example, Gore-Tex®, Dacron®, or polypropylene. As with the previous processes, the first step is to identify the device requirements as indicated in operation 902. Once the desired properties of the finished SMP-integrated fabric device are determined, the appropriate material properties of the SMP strands or threads may be determined as indicated in operation 904. Further, the material properties of the medical fabric thread must be determined and an appropriate medical fabric thread may be selected as indicated in operation 906. Once the SMP thread and the medical fabric thread are selected, they may be woven together to form a fabric as indicated in operation 908. The completed woven fabric may then be formed or constrained into an appropriate deployed shape for the SMP-integrated medical fabric device as indicated in operation 910. Once appropriately formed, the SMP-integrated medical fabric may be subjected to ultraviolet light to cure the SMP-integrated fabric into the deployed shape as indicated in operation 912. The completed SMP-integrated medical fabric device may then be sterilized as indicated in operation 914 and tested to determine whether the completed SMP-integrated medical fabric device meets the desired requirements as indicated in operation 916. If the requirements have been met, the woven SMP-integrated fabric device may be mechanically deformed for storage and later delivery during a medical procedure. Alternatively, if the device requirements set forth in operation 902 have not been met, the manufacturing process 900 may return to step 904 for selection of alternative shape memory polymer formulations or medical device fabrics for integration to form the desired SMP-integrated fabric device.

All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present invention, and do not create limitations, particularly as to the position, orientation, or use of the invention. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. The exemplary drawings are for purposes of illustration only and the dimensions, positions, order and relative sizes reflected in the drawings attached hereto may vary.

The above specification, examples and data provide a complete description of the structure and use of exemplary embodiments of the invention. Although various embodiments of the invention have been described above with a certain degree of particularity, or with reference to one or more individual embodiments, those skilled in the art could make numerous alterations to the disclosed embodiments without departing from the spirit or scope of this invention. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

What is claimed is:

1. A shape memory polymer integrated medical fabric for use in a surgical procedure, the integrated fabric comprising
a medical fabric;
a shape memory polymer integrated with the medical fabric to provide a deformable and reformable structure to the integrated fabric upon placement in vivo.

2. The integrated medical fabric of claim 1, wherein the shape memory polymer is integrated with the medical fabric in a pattern that leaves portions of the medical fabric uncoated.

3. The integrated medical fabric of claim 2, wherein the pattern comprises a border of uncoated medical fabric, said border providing at least one suture attachment point in the surgical procedure.

4. The integrated medical fabric of claim 2, wherein the pattern comprises shape memory polymer integrated fabric on at least one side of the medical fabric.

5. The integrated medical fabric of claim 2, wherein the pattern comprises a border of shape memory integrated fabric surrounding an area of uncoated medical fabric.

6. The integrated medical fabric of claim 1, wherein the shape memory polymer is further selected to have a formulation to achieve a desired rubbery modulus.

7. The integrated medical fabric of claim 1, wherein the shape memory polymer is further selected to have a formulation to achieve a desired activation period.

8. The integrated medical fabric of claim 1, wherein the shape memory polymer is further selected to have a formulation to achieve a desired activation temperature.

9. The integrated medical fabric of claim 1, wherein the surgical procedure is repair of a hernia and the integrated medical fabric is a hernia repair patch.

10. The integrated medical fabric of claim 1, wherein the shape memory polymer comprises thiol and/or vinyl monomers or oligomers.

11. The integrated medical fabric of claim 10, wherein the shape memory polymer further comprises acrylate or methacrylate functional groups.

12. The integrated medical fabric of claim 1, wherein the shape memory polymer is 10 wt % PEGDMA with a Mₙ=1000 and remainder tert-butyl acrylate with 0.1 wt % photoinitiator (2,2 dimethoxy-2-phenylacetopenone).

13. A method of forming a shape memory polymer-integrated fabric comprising
providing a medical fabric;
placing the medical fabric in a mold gasket;
applying a shape memory polymer of a desired formulation to a surface of the medical fabric;
placing a pair of transparent slides on each side of the mold gasket to retain the shape memory polymer against the medical fabric;
exposing the shape memory polymer to ultraviolet light to cure the shape memory polymer; and
releasing the integrated medical fabric with the cured shape memory polymer from the mold gasket.

14. The method of claim 13, further comprising placing a mask on the medical fabric before applying the shape memory polymer to prevent the shape memory polymer from integrating with certain portions of the medical fabric covered by the mask.

15. The method of claim 13, further comprising placing a wax layer adjacent to the medical fabric before applying the shape memory polymer to prevent the shape memory polymer from integrating with certain portions of the medical fabric adjacent to the medical fabric.

16. The method of claim 13, further comprising sterilizing the SMP-integrated medical fabric by exposure to steam or by chemical cleansing after release from the mold gasket.

17. The method of claim 13, wherein the shape memory polymer comprises thiol and/or vinyl monomers or oligomers.

18. The method of claim 17, wherein the shape memory polymer further comprises acrylate or methacrylate functional groups.

19. A molding apparatus for forming a shape memory polymer-integrated medical fabric, the apparatus comprising
at least two molding gaskets operably attached to opposing sides of a medical fabric;
a pair of transparent slides in retaining engagement with a shape memory polymer disposed about the medical fabric; and
an ultraviolet light source configured to cure the shape memory polymer disposed about the medical fabric to create a shape memory polymer integrated medical fabric.

20. The molding apparatus of claim 19, further comprising a mask operably engaged with the medical fabric to prevent the shape memory polymer from integrating with certain portions of the medical fabric covered by the mask.

21. The molding apparatus of claim 19, further comprising a wax layer disposed adjacent to the medical fabric to prevent the shape memory polymer from integrating with certain portions of the medical fabric adjacent to the wax layer.

22. The molding apparatus of claim 21, wherein the shape memory polymer comprises thiol and/or vinyl monomers or oligomers.

23. The molding apparatus of claim 22, wherein the shape memory polymer further comprises acrylate or methacrylate functional groups.
